(19) United States
(12) Patent Application Publication
(10) Pub. No.: US 2004/0147040 A1
(43) Pub. Date: Jul. 29, 2004

(54) Multidimensional separation of biosubstance mixtures for mass spectrometric analysis

(75) Inventors: Martin Bluggel, Dortmund (DE); Martin Schurenberg, Tarmstedt (DE)

Correspondence Address: KUDIRKA & JOBSE, LLP, ONE STATE STREET, SUITE 800, BOSTON, MA, 02109, US

(73) Assignee: Bruker Daltonik GMBH, Bremen (DE)

(21) Appl. No.: 10/685,307

(22) Filed: Oct. 14, 2003

(30) Foreign Application Priority Data
Oct. 14, 2002 (DE) 102 47 896.1

(51) Int. Cl. G01N 1/18

(52) U.S. Cl. 436/173; 436/177

(57) ABSTRACT

The invention relates to methods and instrumentation for two or multidimensional separations of biosubstance mixtures, especially protein mixtures, for mass spectrometric analysis. The invention consists in utilizing the separating effects which are attainable through the selective binding of biosubstances from solutions on solid surfaces with suitable binding characteristics or through the dissolution in fractions of surface-bound biosubstances by means of specially formulated solvent mixtures. According to the invention, these separating effects can be applied for the separation into multiple fractions, and two or more generations of such separations with different separating dimensions can be used in succession to separate the respective fractions into several sub-fractions. These separations can be carried out automatically using pipetting robots without the need for chromatographs and require far less time than multidimensional chromatography or electrophoresis.

### FIELD OF THE INVENTION

The invention relates to methods and instrumentation for multidimensional separations of biosubstance mixtures, especially protein mixtures, for mass spectrometric analysis.

### BACKGROUND OF THE INVENTION

The analysis of complex biosubstance mixtures is becoming increasingly important in the fields of biochemistry, molecular biology, cell research, pharmacological drug development, as well as in preclinical and clinical medical research. The biosubstance mixtures may be protein mixtures, including protein conjugates such as glycoproteins or lipoproteins, or other biosubstances, such as DNA, polysaccharides, metabolites or regulatory substances such as corticosteroids. In the case of proteins, the mixture can be a complete proteome which contains all the proteins from a cell aggregate or a body fluid. In addition, partial proteomes may be obtained from a proteome through any type of coarse fractionation, for example a separation through centrifugation or through broad-spectrum affinity extraction. Of equal interest are synthetically manufactured mixtures such as a mixture of two identical partial proteomes whose cell aggregates are subject to different stress conditions (illness, nutrition, temperature, pharmacological compounds) and whose proteins were labeled differently in order to be able to determine their origin when they are used in mass spectrometric analysis.

The goal of the analysis can either be a simple identification of the biosubstances in the mixture or the determination of the relative frequency of occurrence in comparison to a standard, for example, for diagnostic purposes. In the case of proteins, it may be of interest to search for structurally different proteins which developed through, for example, mutational, post-translational or regulatory changes. Differential expression analyses are frequently applied for investigating the dependency of the formed protein concentrations on stress situations.

The mass spectrometric analysis always requires a preceding separation of the biosubstances, for example using chromatography or electrophoresis such that an easily interpretable mass spectrum results. In the case of proteins, the analysis is sometimes carried out after separation of the unaltered proteins, especially when dealing with smaller proteins ranging up to a maximum of approx. 50 amino acids. If the mixture contains larger proteins, an enzymatic digestion is preferably carried out before the mass spectrometric analysis. In principle, the digestion can take place before or after the chromatographic or electrophoretic separation. Other biosubstances can also be subjected to enzymatic or chemical splitting.

With proteins, a chromatographic or electrophoretic separation before enzymatic digestion has an invaluable advantage: the relationship between the digestion peptides and a protein is known. Thus, the precisely measured masses of the digestion peptides alone produce a pattern which can be used for identification purposes in protein sequence databases on the basis of a virtual digestion of the database proteins. Only when these results are ambiguous must fragmentation spectra be obtained in order to confirm the identification. If the separation is carried out after the enzymatic digestion, a fragmentation measurement is always necessary to identify the digestion peptide. However, separation after the digestion has the major advantage that standard analytical methods can be developed—methods which are practically independent from the type of protein mixture used—and can be as easily applied for membrane proteins as for peptides from body fluids.

Today, 2D gel electrophoresis is generally used when complex mixtures of proteins are to be separated before an enzymatic digestion. For this purpose, SDS-PAGE is normally employed (polyacrylamide gel electrophoresis with sodium dodecylsulfate as solubilizer for the proteins). In one direction, the proteins are separated (without SDS) based on their isoelectric point (the mean ionic charge during protein dissociation in aqueous solutions) in a fixed pH gradient. In the other direction (with SDS), the proteins are separated based on size differences which result in varying electrophoretic migration rates through the gel. This migration rate is, to a large extent, proportional to the protein molecular mass.

For the mass spectrometric analysis, the proteins in the gel are stained, the stained gel pieces are punched out, the proteins are digested in the gel pieces by enzymes—usually by trypsin, the resulting digestion peptides are extracted and the exact masses and sequence of these extracts are measured using mass spectrometry. Commercial robot stations are available for cutting out and digestion.

The established standard measurement methods for the analysis of digestion peptide mixtures are mass spectrometry combined with ionization through either matrix-assisted laser desorption (MALDI=Matrix-Assisted Laser Desorption and Ionization) or electrospray ionization (ESI). For this purpose, Time-of-Flight Mass Spectrometers (TOF-MS) are most often used although Fourier-Transform Ion Cyclotron Resonance Spectrometers (FT-ICR) or high-frequency quadrupole ion trap mass spectrometers (for short: ion trap) can also be utilized. All three types of mass spectrometer produce fragmentation spectra of selected primary ions. These can be used to unambiguously identify the proteins by means of sequence databases, or the peptide structure sequence can be completely or at least partially derived.

It has been found that poor separations can also lead to good results: if two, three or more non-separated proteins (whose digestion peptides mix) are contained in a punched out gel piece, these peptides can also be accurately identified, even if no fragmentation spectra are obtained. The quality and clarity of the identification is strongly dependent on the accuracy of the mass determination, which today is extremely good with several of the aforementioned mass spectrometry methods. Fragmentation spectra drastically improve the identification process again: six to eight overlapping proteins can be determined and identified. In principle, this method is only limited by the consumption of the substance during the acquisition of many fragmentation spectra and by increasing overlapping of isotope groups in a single digestion peptide. For the acquisition of suitable fragmentation spectra, a data-independent feedback control is starting to be employed today in order to only obtain spectra from those peptides whose origin from proteins is not yet known.

However, 2D gel electrophoresis is difficult to handle and does not always generate reproducible results, even in experienced laboratories. The method is generally time-consuming, lasting several days. For this reason, the search for an alternative method to replace 2D gel electrophoresis has been ongoing for a long time.

A much used method is one where the mixture proteins are enzymatically digested before separation and then the digestion peptides are subjected to separation. For this process, 2D chromatography is usually used. With this method, the now very complex mixture consisting of thousands of digestion peptides initially undergoes a separation into hundreds of fractions using liquid chromatography with an initial column material. The individual fractions are then further separated using other column materials and introduced to the mass spectrometer. The second chromatographic separation runs are generally carried out using a coupled LC-MS process with ionization through electrospraying, which enables the fragmentation spectra of the individual peptides to be automatically recorded. From the plethora of peptide fragmentation spectra, the original proteins are digitally reconstructed based on their relationship in protein sequence databases. However, this method also has the disadvantage of long measurement times since each of the chromatographic processes requires approx. thirty minutes (or even much longer). The same applies when chromatography and capillary electrophoresis are coupled.

Numerous studies are concerned with reducing the vast number of digestion peptides. For example, they concentrate on isolating just one digestion peptide at a time from each original protein. These studies will not be discussed further here.

There is also a fundamentally different approach for the separation of protein mixtures which, until now, has rarely been utilized in mass spectrometric analysis. This method involves the separation of selective bonds, as is used especially in multiple assays with the aid of array chips. In the individual fields of the array chips, coatings are deposited, such as antibody or DNA coatings, which can selectively bind individual proteins. During the manufacture of array chips, the aim is to produce coatings which are as highly selective as possible such that only one protein is bound. This is not always successful since all coatings will more or less also bind other proteins. It can therefore be seen that there exists a wide range of coatings with a broad spectrum of selectivity, apart from those coatings which bind all proteins and those which only bind a single type of protein. Up to now, this wide range of coatings has scarcely been used for separation.

### SUMMARY OF THE INVENTION

The present invention provides a method for a rapid, multidimensional separation of the substances in a biosubstance mixture, preferably a protein mixture, and supplies the necessary instrumentation and chemicals in a simple and manageable assembly. The separation should occur automatically and within a few hours. Separating effects are used which are attainable through the selective binding of biosubstances, such as proteins, from solutions on solid surfaces with suitable binding characteristics or through the dissolution in fractions of biosubstances bound to the surface by means of specially formulated solvent mixtures. According to the invention, these separating effects can be applied for the separation into multiple fractions, and two or more generations of such separations with different separating dimensions can be used in succession to separate the respective fractions into several sub-fractions. These separations are based on the respective stationary equilibrium between the surface binding of biosubstances and their dissolution in a solvent. These separations can be carried out automatically using pipetting robots without the need for chromatographs and require far less time than multidimensional chromatography or electrophoresis. Practically all solid phases used in liquid chromatography, but also selectively binding coatings such as those containing antibodies and generally affinitive coatings, can be used as reversibly surface-binding materials. The binding surfaces can be coatings on suspended spheres, preferably magnetic spheres, but also coatings on vessel walls, for example the microvessels in microtitration plates or a spot coating of sample support plates such as MALDI sample support plates.

It is therefore possible to combine steps involving solvent-dependent fractionating detachment of proteins bound to the surface and steps for selective surface binding from solutions. However, the mixing of these different methods is not a requirement for the invention.

In contrast to the dynamic separation of substance mixtures in chromatography with mobile liquid phases where the separation ability is demonstrated using hundreds or even thousands of theoretical plates, fractionating detachment makes use of stationary solution equilibria which only have a separation ability of several tens of theoretical plates. However, several fractions can be obtained through fractionating solution with gradually adjusted solvents. This can be achieved using, for example, solvents with increasing salt concentrations for binding on ion exchangers or increasing concentrations of organic solvents in aqueous solution for binding on reverse phases. The separation is improved through multiple washings of a bound protein fraction with fresh solvent of the same concentration. For this type of separation, the solid phases normally used in chromatography are employed. The physical dimensions of the separation may concern the molecule size, as in size exclusion chromatography, the charge dissociation of molecules in solution, as in ion exchange chromatography or the molecule hydrophobicity, as in reverse phase chromatography.

Furthermore, selectively binding surfaces can be used, which are rarely applied in chromatography precisely because of their more or less strong selectivity. In borderline cases, a surface bound antibody can extract a single type of protein molecule from a particular fraction and for this purpose, broad-spectrum selective surfaces are of special importance. The physical dimension of the separation is determined by the general surface characteristics of the molecule, especially, for instance, separation based on the surface patterns of the molecule can be achieved. These include, for example, the orientation of certain amino acids to one another on the surface of a folded protein or just a short series of amino acids on an unfolded peptide. With proteins, a fraction which was obtained in a previous step is then successively introduced to several selectively binding surfaces where, in each case, a portion of the proteins will be selectively bound. After removing all of the selectively bound proteins from the layers used, the residual solution of the fraction may still contain a portion of the proteins, which can then be used for further analysis. On the other hand, the selectively bound proteins can either be dissolved collectively or fractionated again through, for example, an increasing amount of organic solvents in aqueous solution.

Further fractionations can then occur in other separation dimensions. The resulting end product is a multidimensional matrix of various fractions. In each case, the complexity of the mixture is significantly reduced compared to the original mixture, such that the fractions can be analyzed without any difficulty using mass spectrometry.

An enzymatic digestion of the separated proteins can be included before or after each separation dimension. The digestion can be carried out in free solution or while the proteins are affinitively bound. In general, the digestion peptides must be washed after the digestion, although the washing stage can also be combined with the sample preparation on a MALDI sample support.

The binding layers can be bound to vessel walls or surface spots, but also on small particles. On one hand, the particles can be small, suspended spheres or small particles of another shape (ranging from 50 nanometers to 100 micrometers in diameter) with a corresponding surface coating, preferably a porous one. The spheres may possess a magnetic core such that they can be held to vessel walls by known means or periodically dragged through washing liquids. On the other hand, the small particles can also be larger (with volumes ranging from 0.01 to 1 cubic millimeter) and made from open-pore solid foams or smaller sintered particles. Both the solid foams and the spheres can be used as freely moving particles in liquids (e.g., suspensions) or as surface-bound material.

The procedural steps are preferably carried out using pipetting or dispensing robots. These steps may take place, for example, on microtitration plates and in the final stage, the separated proteins can be prepared on MALDI sample support plates. Ready-made microtitration plates may contain all particle suspensions used, wall coatings, washing liquid, solutions with adjusted salt concentrations, enzymes, buffer solutions to activate the enzymes, samples with mass references and matrix solutions for sample preparation for the ionization by matrix-assisted laser desorption. The solutions with increasing levels of organic solvents, such as methanol or acetonitrile, can be produced by the pipetting robot itself. The microtitration plates, possibly together with other consumables such as pipette tips, could be made commercially available in ‘ready-to-use’ disposable kits.

According to the invention, the method can naturally be combined with other separation techniques such as chromatography or electrophoresis which precede or follow the invention method.

### BRIEF DESCRIPTION OF THE DRAWINGS

The above and further advantages of the invention may be better understood by referring to the following description in conjunction with the accompanying drawings in which:

FIG. 1 shows a cross-section of a microtitration plate (1) with eight microvessels (2) each of which have a wall coating (3) from a suitable surface-binding material in the lower, conical part.

FIG. 2 shows one of these microvessels (2) with an inserted pipette tip (4, 5, 6, 7) of a pipetting robot. The disposable pipette tip has a shaft (4) with a cannula (7) and a specially formed conical tip (5) which is adapted so well to the microvessel cone that only a narrow gap remains between the wall coating (3) and the conical tip (5). Through multiple suctions and ejections, good contact is established between the wall coating (3) and the liquid in the pipette tip such that washing, selective binding and selective solution procedures all occur particularly effectively. The conical tip has small protrusions (6) which rest against the wall and adjust the conical tip so that even minute amounts of liquid can be re-suctioned from the microvessel.

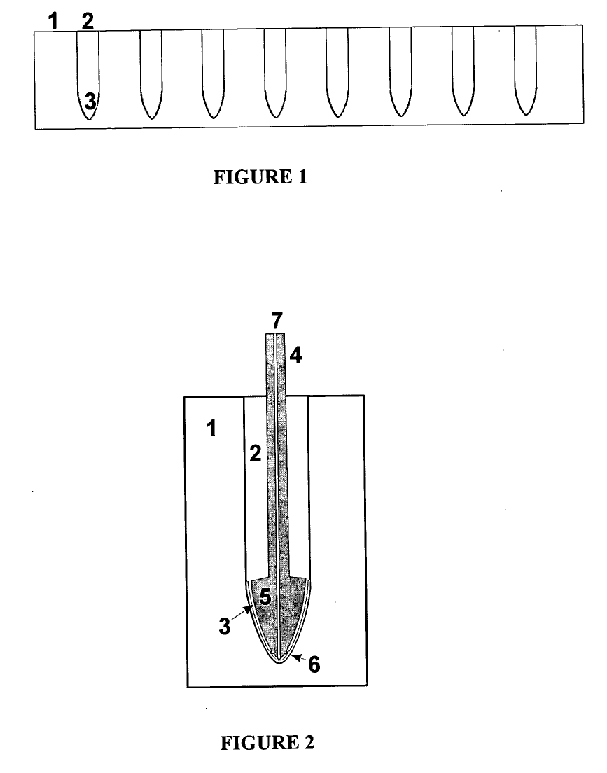

### DETAILED DESCRIPTION

The invention involves carrying out an initial discrete n-fold fractionation of the sample substance mixture and at least a second discrete m-fold fractionation of another separation dimension for each (or only several) of the n fractions. The nxm-fold (or less) fractionated samples are analyzed using mass spectrometry either with further fractionation through other separation methods or with an enzymatic digestion (before or after). Mass spectrometers with the means to measure fragmentation spectra are preferably used.

A preferred method uses a pipetting robot and ready-made microtitration plates sealed with adhesive foils. These plates contain the necessary wall coatings or particle suspensions, washing liquid, extraction liquids with adjusted salt concentrations, enzymes, buffer solutions to activate the enzymes and matrix solutions for preparing the sample for ionization by matrix-assisted laser desorption. The sealing foils can be punctured by the pipette tips or removed before use. The aqueous solutions with increasing levels of organic solvents, such as acetonitrile, can be produced by the pipetting robot itself. The pipetting robot can likewise carry out the necessary steps for MALDI preparations of the separated proteins on appropriate sample support plates, the sample support plates preferably being the same size as the microtitration plates.

Using microtitration plates, a method according to the invention for a selected protein mixture can consist of the following steps. As an example, for the method presented here with two separation dimensions, a first separation dimension through size exclusion is initially performed. Size exclusion chromatography is not a part of the method according to the invention since no surface binding is involved. The combined method thus has three separation dimensions, of which the final two belong to the method according to the invention:

The steps of an example method are as follows:

(1) the protein mixture is separated into eight fractions according to molecular size using a size exclusion microcolumn. A pipetting robot can easily be used for implementing this separation with a microcolumn.

(2) The eight fractions obtained through the first dimension of the combined method are collected in eight microvessels whose wall surfaces are coated with ion exchange material where the proteins are ionically bound.

(3) The protein mixtures bound in the ion exchangers are repeatedly washed with a washing liquid which does not dissolve the protein.

(4) In the first separation dimension of the method according to the invention (i.e., the second dimension of the combined method), the proteins are extracted in six stages using aqueous salt solutions of increasing concentration (for example, ammonium chloride). Each group of six fractions (in total 48 fractions) is collected in microvessels which are coated with affinity absorbing reverse phases where the proteins are affinitively bound. The proteins are now roughly sorted according to molecule size and charge, which is also the basic principle for 2D gel electrophoresis.

(5) Washing liquids are selected which will not dissolve the proteins and are used to remove salts from each of the proteins from all 48 fractions.

(6) In the second separation dimension of the method according to the invention (i.e., the third separation dimension of the combined method), each of the proteins is dissolved in eight stages using solvent mixtures which contain increasing concentrations of organic solvents (for example, acetonitrile) in aqueous solution. The proteins are thus separated according to their hydrophobicity, as in reverse phase chromatography.

(7) The now 384 fractions are transferred to a MALDI sample support plate which is already coated with a thin matrix layer intended for the affinitive binding of the proteins.

(8) The sample support plate is transferred in the usual way to a time-of-flight mass spectrometer for analysis, although an optional washing phase for the sample support plate can be interposed.

A pipetting robot can be easily be implemented and automated for this method. A favorable option is a pipetting robot with eight pipetting needles such that eight separation and washing stages can be carried out simultaneously. In this case, automation is more easily implemented than with other types of separation methods.

Of course, the method can be restricted so that only the fraction of light peptides obtained with size exclusion chromatography is subjected to the following steps of the method according to the invention. These peptides do not require an enzymatic digestion for their analysis. However, an enzymatic digestion may follow step (6), for example with trypsin. In this case, all proteins can be subjected to the digestion or only the larger molecules which were already separated from the smaller peptides in step (1).

For the steps of the procedure according to the invention, a practical microtitration plate is used with 96 microvessels, of which eight are coated with ion exchangers on the inner walls and 48 with reverse phases. The microvessels can be sealed with a foil which can be pierced. Both ion exchange materials and reverse phases can be the same in the respective microvessels or adjusted for a preceding separation according to molecule size. The remaining microvessels of this microtitration plate (or of others) may contain washing liquids, extraction liquids, enzymes, activating buffers for the enzymes or other such substances. A pipetting robot with eight pipetting needles is preferably used. The method can then be carried out in approximately two hours (excluding a tryptic digestion).

The wall-coated microvessels can have a special, conical shape at the bottom which is adjusted for the shape of the disposable pipette tips as shown in FIG. 2. The matching shapes ensure a good, flowing contact between the washing or elution liquids and the wall coatings, and with multiple suction and ejection of the liquid, a particularly effective washing or dissolving effect results. Pipette tip protrusions prevent the tip from completely resting against the wall. Pipette tips of this form can also practically remove liquids completely.

Another embodiment of the invention does not make use of the microvessel wall coatings, but rather suspensions of tiny particles with surface coatings, as are already commercially available, preferably as magnetic spheres. The magnetic spheres can have diameters ranging from 50 nanometers to approximately 10 micrometers. For this application, the special features are incorporated into the pipetting robot such that, through movable magnets, the spheres can be dragged slowly back and forth through the liquid several times or can be held on the wall in order to remove the liquid.

Furthermore, instead of being used in a suspension, the small particles can be deposited as a thin film on a flat surface in a strongly hydrophobic environment. In this way, particles with larger diameters or non-magnetizable particles can also be used. For example, they could be adhered to the surface. The washing and dissolving liquids are then simply pipetted as drops onto the surface or dispensed with piezo dispensers.

“Small particles” includes all general materials with a large active external or internal surfaces. On one hand, these particles can be small, suspended spheres or small particles of another shape (ranging from 5 to 100 micrometers in diameter) with a correspondingly activated surface coating, preferably a porous one. The spheres may possess a magnetic core such that they can be held to vessel walls by known means or dragged through washing liquid. On the other hand, the small particles can also be larger (with volumes ranging from 0.01 to 1 cubic millimeter) and made from open-pored solid foams or smaller sintered particles. Both the solid foams and the spheres can be used as freely moving particles in liquids (e.g. suspensions), as surface-bound material or as enclosed in microcolumns or pipette tips.

Until now, spongy microspheres from reverse phase materials (Poros, ABI Bio-Systems) with pipette tips filled with spongy reverse phase material (ZipTips, Millipore) or magnetic spheres with C18 coatings have proven especially successful for the purification of peptide, protein or DNA mixtures. These materials bind peptides or oligonucleotides through hydrophobic bonds. In general, the biomolecules can be eluted with aqueous methanol or acetonitrile solutions; the elution takes places in stages either with adjusted concentrations of organic solvents or through adjusted pH values at each stage. All of these materials can be used within the framework of the separation of substances according to the invention.

The separation ability of solvent equilibria is only moderate. However, it can be improved through multiple solution steps with fresh solvent of an identical concentration (or even a somewhat weaker one). The proteins are repeatedly extracted from the ion exchangers with the salt solutions of the same concentration (or with salt solutions of slightly decreasing concentrations) in order to flush out all those proteins which are exchangeable at this concentration. In the case of reverse phases, the proteins are repeatedly washed out with the same aqueous acetonitrile solution. Thus, a more sharply defined separation of the proteins results.

By using the pipette tips shown in FIG. 2, proteins can also be dynamically washed out using a solution with a specific concentration. The solution is first simply placed in a microvessel which carries proteins in the wall coating; ensuring that the pipette tip does not contact the ejected liquid. In this way, only a very small portion of the proteins go into solution. Then the pipette tip is lowered; the practically still clean solution is now located to a large extent above the wall coating. The solution is then very slowly suctioned off establishing a good contact between the wall coating and solution. Thus the coating is always dynamically washed with a clean solution resulting in a high separation resolution of the individual fractions.

If a separation method has a resolution of 40 theoretical plates—which can certainly be achieved with this separation method—then 80% of the proteins are found in a single fraction from a fractionation into eight fractions. Only 20% of the proteins are found in two neighboring fractions and with thoroughly different distribution ratios. For two successive separation generations with the same selectivity and, in each case, involving eight fractions, 64% of the proteins are found in only one fraction, 32% are distributed over two fractions and 4% are spread over four fractions. For three separation generations, 50% of the proteins are found in only one of the now 512 fractions, approx. 37% are spread over two fractions, approx. 12% are distributed over four fractions and less than a percent of the proteins is distributed over eight fractions. In the least favorable case, the latter is evenly distributed, and in the most favorable case, one of these fractions contains the greater portion of the proteins. These numbers mirror the high separation potential of the invention.

Frequently, not all proteins are investigated, rather the objective is only the analysis of a protein subset. One example is the study of the relative expression intensity of certain proteins in stressed and unstressed tissue. If this subset is small, then all separation steps do not necessarily need to be carried out. In addition, by shifting the separation limits, it is then possible to ensure that the desired proteins are only found in one fraction.

The two most favorable and so far most investigated separation dimensions for this method are molecular charging measured by ion exchangers and molecular hydrophobicity measured by reverse phases. Until now, the separation through other types of affinities has been studied to a lesser degree despite their high potential.

A separation by ion exchangers is carried out by extracting the bound proteins with salt solutions of gradually increasing concentration. Alkali-free salts should be used for later MALDI analysis. Ammonium chloride (NH₄Cl) has proven to be an excellent choice for this extraction. The concentration increments are preferably selected in such a way that, for the particular analytical problem, the same number of proteins are separated in each separation step, as far as possible. The salt concentrations can be determined in calibration experiments. Multiple washing out with solutions of the same concentration increases the separation selectivity. The separated proteins in the salt solutions are then placed in microvessels with reverse phases, where the proteins are affinitively bound. In this way, proteins can also be consecutively removed from larger quantities of salt solutions.

The proteins on the reverse phases are washed again, this time with a liquid which does not elute the proteins from the reverse phases. For example, acidified water can be used for this washing. Here too, microvessel wall coatings with reverse phases or reverse phase beads with magnetic cores can be used. The washing time can be shortened using magnets or with the help of special pipette tips. The magnets also serve to hold the particles to the wall of the wells so that the washing liquid can be completely removed from the wells of the microfiltration plates without removing the particles containing the bound proteins.

The separation stage with the reverse phases consists of a partial protein extraction with an aqueous organic solvent solution where the organic solvent concentration gradually increases for the different fractions. Multiple washing out with solutions of the same concentration can also be carried out here in order to increase the separation selectivity. The protein solutions can, for example, be directly deposited onto MALDI sample support plates which are already coated with a thin layer of α-cyano-4-hydroxy-cinnamic acid. This layer affinitively binds the proteins and the solution can be suctioned off after a short exposure time.

The separation with partially selective affinity phases occurs differently than with the previously described phases. In this case, each fraction of the above-mentioned separations is successively introduced to the different affinity phases, which can be wall coatings in microvessels, for example. Each of these affinity phases extracts particular proteins from the fraction and binds it. After the solution has been introduced to a series of, for example, seven different affinity phases, the remaining solution may still contain proteins which did not bind with any affinity phase. This remainder then forms an eighth fraction which is transferred, for example, to a broad-spectrum reverse phase to ensure that the proteins are likewise surface-bound.

From these partially selective affinity phases, the proteins can be either completely extracted using appropriate solvents or fractionated by means of increasing organic solvent concentrations. In the latter case, a separation with two dimensions is achieved with the partially selective affinity phases.

The microtitration plates may contain, ready-made and sealed, all the particle suspensions used, wall coatings, washing liquids, solutions with adjusted salt contents, enzymes and buffer solutions to activate the enzymes. The microtitration plates, possibly together with other consumables such as specially shaped pipette tips could be made commercially available in ‘ready-to-use’ disposable kits.

The method according to the invention can be varied in numerous ways by a specialist with a full understanding of the fundamental principles. The invention is therefore not restricted to the aforementioned examples. Other separation methods (with other dimensions) can especially be used, other types of digestions or splitting can be applied or the separations can be carried out using small continuous-flow columns filled with spongy phases.

What is claimed is:

1. Method for the multidimensional separation of a biosubstance mixture for mass spectrometric analysis, the method comprising separating the biosubstance mixture into multiple discrete fractions using a fractionating binding of biosubstances from the mixture on solid surfaces or a fractionating dissolution of biosubstances from surface-bound biosubstance mixtures through specially adjusted solvent, wherein two or more separation generations with different separating dimensions are used to successively separate the respective fractions into multiple discrete sub-fractions.

2. Method according to claim 1, wherein the biosubstance mixture is a mixture of proteins and/or protein conjugates.

3. Method according to claim 1 wherein the surface binding is carried out with liquid chromatography phases or substance-affinitive layers.

4. Method according to claim 1 wherein the surface binding occurs in microtitration plates having microvessels coated on the inner walls with substance-binding layers.

5. Method according to claim 1, wherein the surface binding occurs in microtitration plates having microvessels containing suspensions of surface-coated particles.

6. Method according to claim 5, wherein the particles employed are magnetizable spheres.

7. Method according to claim 1, wherein the surface binding occurs on flat supports whose hydrophobic surface is spot coated with substance-binding layers.

8. Method according to claim 1, wherein an enzymatic digestion of the separated biosubstances is carried out before the mass spectrometric analysis.

9. Method according to claim 1, wherein a prior separation of the biosubstance mixture is carried out using size exclusion chromatography.

10. Method according to claim 9, wherein the size exclusion chromatography uses a microcolumn.

11. Method according to claim 1, wherein procedural steps for separating the biosubstances are carried out using a pipetting robot.

12. Method according to claim 11, wherein preparations for mass spectrometric analysis of samples from separated fractions are carried out using MALDI sample support plates as well as a pipetting robot.

13. A microtitration apparatus comprising a microtitration plate having a plurality of microvessels that are sealed with a foil that can be pierced, the microvessels containing at least one of a wall coating with a surface-binding material and a suspension of small particles having a surface-binding material.

14. A microtitration apparatus according to claim 13, wherein the microvessels contain at least one of extraction solutions with adjusted salt contents, a washing liquid and, enzymes and buffer solutions for activating the enzymes.

15. A microtitration apparatus according to claim 13, wherein a matrix substance for sample preparation for ionization by matrix-assisted laser desorption is also contained in the microtitration plate.

16. A microtitration apparatus according to claim 13 further comprising a plurality of pipefte tips shaped to fit within the microvessels.

17. A pipette tip for a pipetting robot that fits within a microvessel of a microtitration plate, the pipette tip having at least one surface protrusion that contacts an inner wall of the microvessel so as to maintain a narrow gap between the pipette tip and the inner wall of the microvessel.
